(19) United States
(12) Patent Application Publication
(10) Pub. No.: US 2022/0118178 A1
(43) Pub. Date: Apr. 21, 2022

(54) IV GRAVITY DELIVERY MONITOR

(71) Applicant: Pneuma Systems Corporation, Portsmouth, NH (US)

(72) Inventors: Jeffrey A. Carlisle, Portsmouth, NH (US); Brent Nibarger, Portsmouth, NH (US)

(21) Appl. No.: 17/506,585

(22) Filed: Oct. 20, 2021

Related U.S. Application Data
(60) Provisional application No. 63/093,905, filed on Oct. 20, 2020.

(51) Int. Cl. A61M 5/168; G16H 20/17; G16H 40/63

(52) U.S. Cl. CPC A61M 5/1689; A61M 5/16854; A61M 2205/3331; G16H 40/63; G16H 20/17

(57) ABSTRACT

A monitoring system for a gravity infusion IV tubing includes a drip chamber having an inlet configured to receive fluid from a fluid source. The drip chamber also has an outlet configured to deliver fluid towards a patient. The system includes a pressure sensor pneumatically coupled with the drip chamber. The pressure sensor is configured to measure the pressure inside the drip chamber. A controller is configured to receive pressure measurements from the pressure sensor and to use the pressure measurements to count a number of drops entering the drip chamber from the fluid source.

### PRIORITY

This patent application claims priority from U.S. provisional patent application No. 63/093,905, filed Oct. 20, 2020, entitled, IV GRAVITY DELIVERY MONITOR, and naming Jeffrey Carlisle as inventor, the disclosure of which is incorporated herein, in its entirety, by reference.

### FIELD OF THE INVENTION

Illustrative embodiments generally relate to intravenous fluid delivery and, more particularly, various embodiments of the invention relate to monitoring and/or managing intravenous fluid delivery using gravity.

### BACKGROUND OF THE INVENTION

Gravity administration of intravenous (IV) fluids is still the most common way to deliver fluids and drugs to patients. The pressure generated by the head height of liquid drives fluid through a tube that is restricted by a proportionally resistive roller clamp. The caregiver manually observes the rate of drop formation into a visible drip chamber, adjusting the position of the roller clamp to achieve the desired frequency of drops.

Several common situations can cause the flow rate to change in a way that can harm the patient. These include, but are not limited to, changes in the position of the roller clamp due to impact or mechanical or thermal drift, changes in the head height of the fluid source, or changes in the intravenous flow resistance. The unpredictable timing of the depletion of the fluid source creates a frequent problem in which air may enter the patient line, sometimes requiring a hazardous re-priming procedure.

The art has responded to these problems by deploying devices that monitor the drops and create alarms when the drop rate has substantially changed. Undesirably, these systems often produce an “alarm fatigue” for caregivers or patients. Flow rate monitors, even when effective, do not adequately monitor the volume of remaining fluid. Monitoring and alarming, by themselves, consequently often do not produce a safer clinical environment.

### SUMMARY OF VARIOUS EMBODIMENTS

In accordance with an embodiment, a monitoring system for a gravity infusion IV tubing includes a drip chamber having an inlet configured to receive fluid from a fluid source. The drip chamber also has an outlet configured to deliver fluid towards a patient. The system includes a pressure sensor pneumatically coupled with the drip chamber. The pressure sensor is configured to measure the pressure inside the drip chamber. A controller is configured to receive pressure measurements from the pressure sensor and to use the pressure measurements to count a number of drops entering the drip chamber from the fluid source.

Among other things, the controller may determine a fluid flow rate into and/or out of the chamber from the pressure measurements. The controller may also determine whether there is an occlusion into and/or out of the chamber from the pressure measurements. To that end, the pressure sensor may have a sensitivity of 1/1000 PSI or better. The controller may be configured to determine a change in relative position of a head height of the fluid in the container relative to the patient. The controller may further be configured to determine a remaining time of the infusion as a function of the remaining liquid to be delivered.

In various embodiments, the controller counts each drop by identifying a repeating cycle of pressure measurements. The repeating cycle includes an increase in pressure followed by a decrease in pressure when the drop breaks off.

Various embodiments may further include an air blocking membrane distal to the drip chamber. The air blocking membrane is configured to mitigate air from passing through the air blocking membrane and entering the patient line. The drip chamber may also have a cover with a pneumatic tube coupling the drip chamber with the pressure sensor.

In accordance with yet another embodiment, a method monitors a gravity infusion IV tubing. The method provides a drip chamber having an inlet configured to receive fluid from a fluid source and an outlet configured to deliver fluid towards a patient. The method also provides a pressure sensor pneumatically coupled with the drip chamber. The pressure sensor is configured to measure the pressure inside the drip chamber. The method also provides a controller configured to receive pressure measurements from the pressure sensor. The method determines a number of drops that enter the drip chamber from the fluid source by detecting a small pressure increase followed by a small pressure decrease.

The method may also determine a flow rate out of the drip chamber as a function of the pressure measurements. The method may provide an alarm when the determined flow rate deviates by more than a pre-determined amount from a selected flow rate. The method may also determine a head height of the drug container. A warning may be provided if an empty drug container condition is imminent. An indication of a time left until the drug container is empty may also be provided

Some embodiments may generate a positive pressure in the drip chamber to prevent further fluid from flowing from the drug container into the drip chamber. Preferably, the positive pressure is low enough not to pass through an air-blocking membrane distal to the drip chamber.

In accordance with one embodiment of the invention, an IV administration set has a source input to receive fluid from a fluid source, a drip chamber having a fluid input and a fluid output, and an N tube coupled between the drip chamber output and the patient. The set also has a pressure system fluidly coupled with the drip chamber. The pressure system is configured to monitor drop formation into the drip chamber and to monitor the pressure of proximal and distal head heights.

The pressure system may use pressure signals to detect and count timing intervals of drops in the drip chamber. For example, the pressure system may use the pressure signals to calculate flow rate of fluid, by way of drop frequency, within the drip chamber. In some embodiments, the set also may have a pressure manager to vary the pressure within the drip chamber. To that end, the pressure manager having a pressure sensor, a pneumatic generator, and valve assembly for selective application of positive or negative pressure to the drip chamber. The pressure manager may use a fluid line to fluidly couple the pressure manager to the drip chamber.

Some embodiments also provide output information for a clinician. For example, the IV administration set may have an output that produces information for informing a user on adjusting a roller clamp coupled with the IV tube. For example, the set may have an alarm that produces output alarm indicia when the actual rate deviates by more than a pre-determined amount from an original set point. Among other things, the output alarm indicia may indicate when the source fluid has reached a critically low head height, providing a warning if an empty container condition is imminent (e.g., 5 minutes, 1 minute, etc.).

The system also may have an air blocking membrane distal to the drip chamber to mitigate air from passing through the air blocking membrane entering the patient line, up to a certain pressure. The pressure sensor also may be configured to apply a positive pressure as a function of detected pressure. This positive pressure preferably is both sufficient to block fluid flow from the source, and low enough to not pass through the air-blocking membrane. In addition, the system may have logic for determining an upstream and/or downstream occlusion.

In some embodiments, a subsystem of a comprehensive flow monitoring system analyzes pressure signals on the order of 1 Hz from a gravity drip chamber to count drop formation and includes computation means to convert that reading into conventional units of fluid flow such as mL per hour. The same pressure signals, when viewed over minutes, can be used to record the head height of the fluid source entering the drip chamber, reflecting the remaining liquid to be delivered and therefore, along with the measured flow rate, can provide the time remaining in the infusion. The combination of flow rate and changing head height can be used to estimate the fluid volume remaining.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A schematically shows a system for fluid delivery in accordance with illustrative embodiments of the invention.

FIG. 1B schematically shows the intravenous (“IV”) administration set in accordance with illustrative embodiments of the invention.

FIGS. 2A, 2B, and 3 schematically shows a drip chamber of FIG. 1.

FIG. 4 schematically shows a chart of pressure signals over time, recorded from pressure within the pneumatic tube.

FIG. 5A schematically shows a screenshot of pressure signals over time recorded from pressure within the pneumatic tube in accordance with illustrative embodiments of the invention.

FIG. 5B schematically shows the conditions of drop formation in the drip chamber of FIG. 2.

FIG. 5C illustrates the flow rate calculation using drop intervals in accordance with illustrative embodiments of the invention.

FIG. 5D illustrates a blown up and idealized view of FIG. 5A between points 501 and 503.

FIG. 5E shows the decomposition of the waveform of FIG. 5D, separately showing pressure changes due to in-flow and pressure changes due to out-flow from the drip chamber.

FIG. 6 shows a close-up view of the drip chamber in accordance with illustrative embodiments of the invention.

FIG. 7 illustrates the pressure response to a downstream occlusion in accordance with illustrative embodiments of the invention.

FIG. 8 illustrates the pressure response to an upstream occlusion in accordance with illustrative embodiments of the invention.

FIG. 9 illustrates the pressure response to the depletion of liquid from a source bag in accordance with illustrative embodiments of the invention.

FIG. 10A schematically shows various components of a control system in accordance with illustrative embodiments of the invention.

FIG. 10 B schematically shows a detailed block diagram of the system in accordance with illustrative embodiments of the invention.

FIG. 11A shows a computational process of the flow rate engine to convert drop detection into a flow rate value in accordance with illustrative embodiments.

FIG. 11B shows an example of the process of FIG. 11A.

FIG. 12A shows a computational process of the volume calculation engine to determine remaining liquid volume in the bag from a flow rate and a head height in the bag in accordance with illustrative embodiments of the invention.

FIG. 12B shows an example of the process of FIG. 12A.

FIG. 13A shows a computational process of the volume calculation engine to determine remaining gas volume in the drip chamber in accordance with illustrative embodiments of the invention.

FIG. 13B shows an example of the process of FIG. 13A FIG. 14A schematically shows a fluid source container having compartments with variable diameter and correspondingly variable surface area in accordance with illustrative embodiments of the invention.

FIG. 14B schematically shows a variable pressure change as fluid leaves the container for the various compartments of FIG. 14A.

### DESCRIPTION OF ILLUSTRATIVE EMBODIMENTS

In illustrative embodiments, a method and/or system monitors fluid flow into a drip chamber and measures the remaining liquid in the fluid source using a pressure measurement from the drip chamber or a tube pneumatically coupled thereto. The pressure measurement provides a significant operational advantage that can significantly improve intravenous delivery results. The system may use the pressure measurement to count the number of drops entering the chamber from a drug container, to determine the flow rate into the chamber, to determine the flow rate exiting the chamber, to determine the fluid remaining in the drug container, and/or to take corrective action in view of any of the above determinations. Details of illustrative embodiments are discussed below.

Various embodiments improve the safety and control of gravity intravenous infusions with a slight adaptation of a common tubing administration set and the precise monitoring of pressure within a drip chamber, providing for automated monitoring and reporting of results. One resultant benefit mitigates common hazards associated with IV gravity infusions.

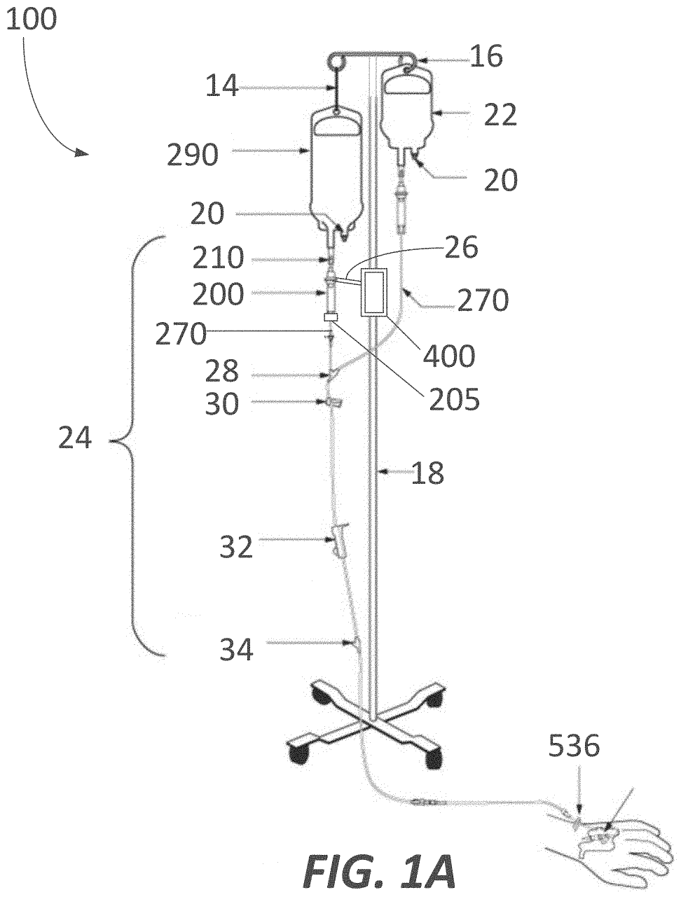

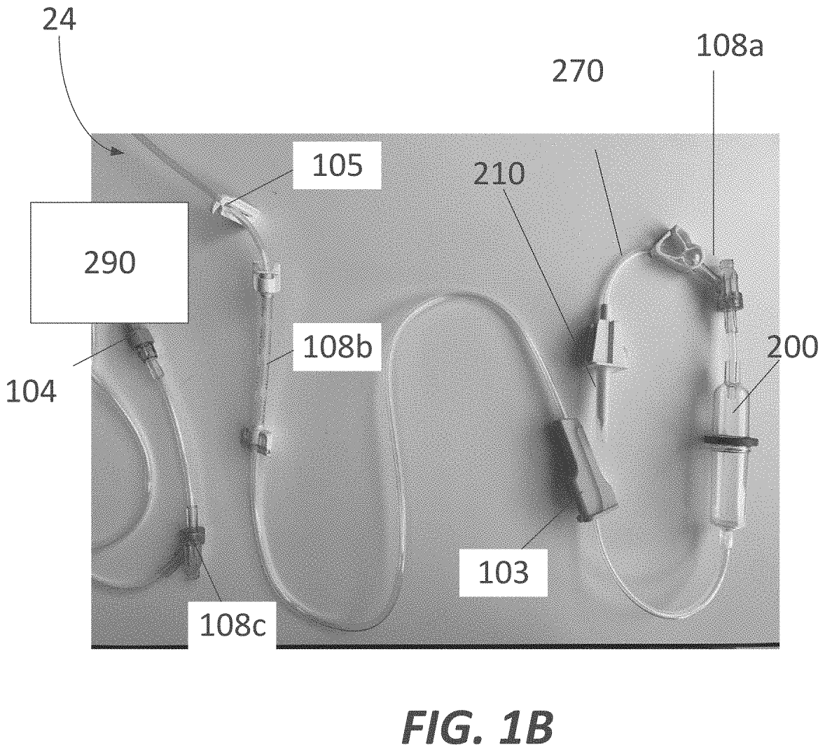

FIG. 1A schematically shows a fluidic system 100 for delivering a drug or other solution (e.g., saline) to a patient 536 in accordance with illustrative embodiments of the invention. The drug may be contained within a fluid container 290, such as primary IV bag 290 hanging from an extender 14 coupled to a hook 16 on an IV pole 18. In some embodiments, the container 290 may have a liquid volume of, for example, 10 mL to 3,000 mL.

The drug may be injected into the IV bag 290 via an injection port 20, prior to or while the IV bag 290 is fluidly coupled with the patient 536. In some embodiments, the system 100 may also include a secondary IV bag 22 also mounted on the IV pole 18. Although FIG. 1A shows the system 100 with a secondary IV bag 22, various embodiments may operate with only a single bag 290. Furthermore, although FIG. 1A shows the bag 290 hanging above the patient 536, illustrative embodiments are able to monitor and detect movement of the bag 290, including below the patient's 536 heart.

The system includes an IV tubing set 24. The tubing set 24 includes a spike 210 configured to fluidly couple the IV tubing set 24 with the drug container 290. In practice, the spike 210 is positioned into a complementary opening in the IV bag or bottle 290.

As known by those in the art, gravity infusion pushes the fluid down through the IV tubing set 24 into the patient's 536 vein. The higher the bag 290 is hung, the greater the gravitational pressure on the IV fluid to go downward through the tubing. If the IV bag 290 is not hung high enough, there is not sufficient pressure caused by gravity to force the fluid into the vein. So, the IV bag 290 preferably is hung above the patient's 536 heart in order for there to be enough pressure for the IV fluid to infuse, and it is standard procedure to hang the IV bag 290 at least 3 feet above an adult patient's 536 heart to ensure there is enough pressure to keep the IV running at a constant rate.

Also, since changing the height of the IV bag 290 changes the gravitational pressure on the fluid, a change in the bag's 290 height over the patient's 536 heart changes the infusion rate of the IV. If the IV bag 290 gets higher above the patient's 536 heart, the IV infusion rate speeds up. On the other hand, if the IV bag 290 gets lower to the patient's 536 heart, the IV infusion rate slows down. Because of this property, if a patient 536 who has been lying down when the IV was set up then sits up, the IV infusion rate slows down because the IV is now closer to the patient's 536 heart. In fact, technically any small movement by the patient or shift in position can change the rate at which the IV is infusing. Because of this, IVs are frequently checked to make sure that they are still infusing at the correct rate; usually once an hour and after any major position change of the patient 536. Illustrative embodiments advantageously provide automated monitoring of the fluid flow rate, and also are able to detect changes in position of the bag 290 relative to the patient 536, enabling fewer checks by practitioners.

The system 100 may also include a slider clamp 30 and/or a roller clamp 32. The roller clamp 32 controls the rate at which the IV fluid infuses. IV medication is ordered to infuse at a specific rate, and one of the major tasks of hospital nurses is to set up the IV so that it infuses at the prescribed rate and to adjust the IV periodically if the rate has changed so that it remains at the ordered rate. The rate at which an IV fluid infuses is referred to as the IV infusion rate or flow rate. Illustrative embodiments deliver a targeted flow rate by using pressure feedback (directly or indirectly) from the drip chamber 200.

As known by those of skill in the art, rolling the roller clamp 32 one way squeezes the IV tubing 270 more tightly, making it more narrow and therefore making the fluid flow through the tubing slower. If the roller is rolled the other way, it loosens its pinching of the IV tubing 270, making the tubing less narrow, and allowing the IV fluid to flow through at a faster rate. For example, if the medical practitioner determines (by looking at the drip chamber and counting drops) that an IV is infusing at a rate of 50 gtt/min, but it was ordered to infuse at a rate of 30 gtt/min, the roller clamp 32 may be tightened to slow the drip rate down until only 30 drops are counted going through the drip chamber 200 each minute.

The tubing set 24 also includes a drip chamber 200. Typically, the drip chamber 200 is formed from a transparent plastic, such that the inside of the drip chamber 200 can be seen by medical staff. However, illustrative embodiments precisely monitor the volume inside the drip chamber 200, and therefore, the drip chamber 200 may be formed from opaque materials. As is known in the art, medical staff measure the speed of a manual IV setup by looking at the drip chamber 200 and counting the number of drops per minute. For example, if 25 drops are counted over the period of 60 seconds, the IV is infusing at a rate of 25 drops per minute, or 25 gtt/min. In reality, medical staff may not count the number of drops in a full minute; instead, they may count the number of drops, for example, over a period of 15 seconds, and then multiply that number by 4 to get the number of drops in a full minute.

Additional details of various parts of the system 100 are disclosed in co-pending and commonly owned U.S. application Ser. No. 17/362,603, which is incorporated herein by reference. For example, various embodiments may include ports 20 on each of the primary IV bag 290 and the secondary IV bag 22, the port 28 below the drip chamber 200 that is connected with the secondary IV bag 22, and an injection port 34 close to where the needle goes into the patient's 536 vein. The injection port 20 on the actual IV bags 290, 22 may be used to mix medication with the fluid that is in the IV bag 290. Medication injected into this port 20 and mixed (e.g., by rolling the bag 290), causes the patient 536 to receive both the medication and the IV fluid at the same time. Some embodiments inject medication or a second kind of IV fluid directly so that it does not mix with the IV fluid bag 290 (e.g., into one of the ports 28 or 34 that are located below the drip chamber 200).

It is common in the art to use a drip chamber for gravity infusion. However, the current state of the art includes a patient 536 safety feedback loop that is the human medical practitioner counting drops and computing a target flow rate. The medical practitioner then uses the roller clamp 32 to adjust downstream resistance. There are well known control problems with the state of the art. For example, the flow rate sometimes unintentionally goes up or down from the target flow rate because of flow rate errors associated with gravity infusions. Advantageously, illustrative embodiments monitor the flow rate and may make adjustments thereto.

Other prior art the inventor is aware of include optical trackers that optically count the number of the drops and therefore the flow rate. Optical trackers generate a lot of unnecessary alarms (e.g., causing alarm fatigue) because the optical trackers are prone to error caused by motion and other artifacts. Furthermore, optical methods are not able to determine whether fluid flow has stopped because the drug deliver container 290 is empty, the drug delivery container 290 is occluded, or if the downstream IV tubing is occluded. Illustrative embodiments advantageously solve all of these problems with the prior art. Accordingly, various embodiments do not require a drop sensor that is separate from the pressure sensor.

Illustrative embodiments pneumatically couple the drip chamber 200 with the controller 400 that controls a pneumatic pressure generator (such as a tightly load coupled pneumatic driver). The medical practitioner may communicate with the controller 400, which may be in a housing 42, via a user interface 44 (e.g., touch screen interface 44). The user interface 44 allows users to select, among other things, a targeted flow rate, and also to set alarm conditions.

As described further below, the controller 400 accurately measures the pressure and calculates a volume of the fluid inside the drip chamber 200. Furthermore, the controller 400 can determine whether the drug container 290 is empty or near empty, a flower rate from the drip chamber 200, and/or whether there is an occlusion in the IV tubing.

In the prior art, generally, the drip chamber 200 is kept about half-full. This is because if the drip chamber 200 is too full, medical practitioners are not able to see the drops to count them, and thus are unable to determine the rate at which the IV is infusing. On the other hand, if the drip chamber 200 is not full enough, then this allows air to get into the output IV tubing 270, which means that air would get into the patient's 536 circulatory system, which could be very dangerous, blocking a blood vessel or stopping the heart. In illustrative embodiments, the controller 400 precisely calculates the flow rate, and therefore, advantageously reduces or eliminates these risks. Furthermore, the controller 400 may detect the volume of any air bubble that makes it into output IV tubing 270.

Illustrative embodiments use pressure sensors that detect minor variations in pressure. For example, the pressure sensor may measure pressure to a 1/1000th of a PSI. The pressure sensors may be coupled with the drip chamber 200 via the pneumatic tube 26 (e.g., the pressure sensors may be in the controller 400 housing).

FIG. 1B shows the administration set 24 in accordance with illustrative embodiments of the invention. The administration set 24 includes many components that are assembled, packaged, labeled, and sterilized for use. Specifically, an IV bag spike 210 connects an IV Tube 270 with a fluid source, which may be a solution or drug mixture. Liquid enters a Drip Chamber 200, which in this example includes a molded feature that facilitates the formation of drops of a fixed size, such as 15 drops per milliliter. The administration set 24 also includes the roller clamp 32, which selectively restricts liquid flow through IV tube 270. The pinch clamp 30 can stop the fluid flow altogether. Fluid leaves the administration set 24 via a luer connector 104 to the patient 536, using standard venous access pathways. Various accessory components, such as those identified by reference numbers 108a, 108b, and 108c, are often configured to administration sets.

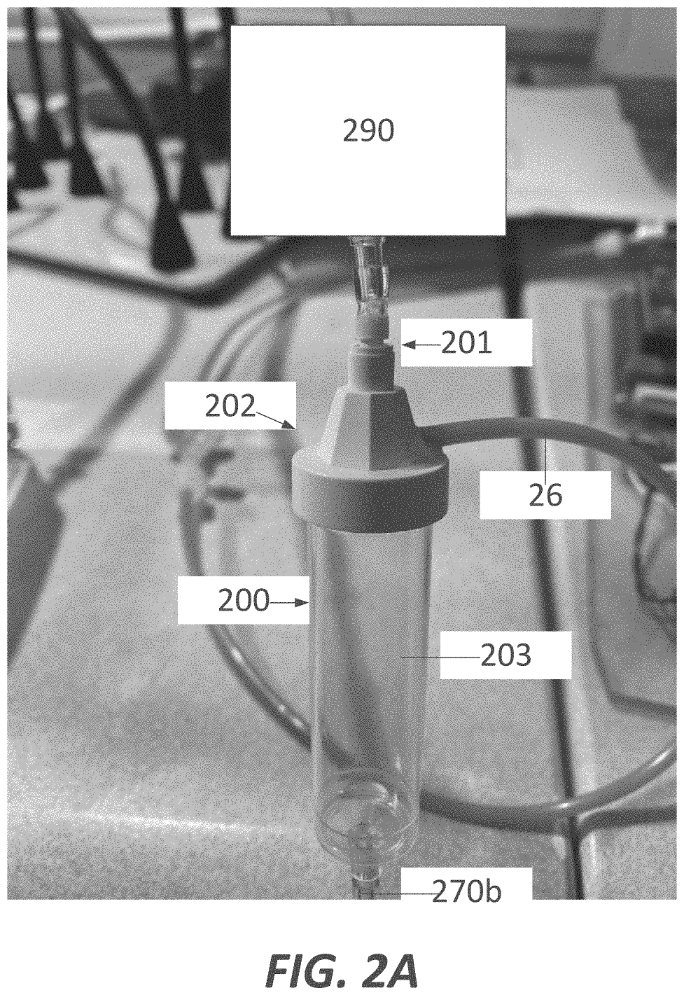

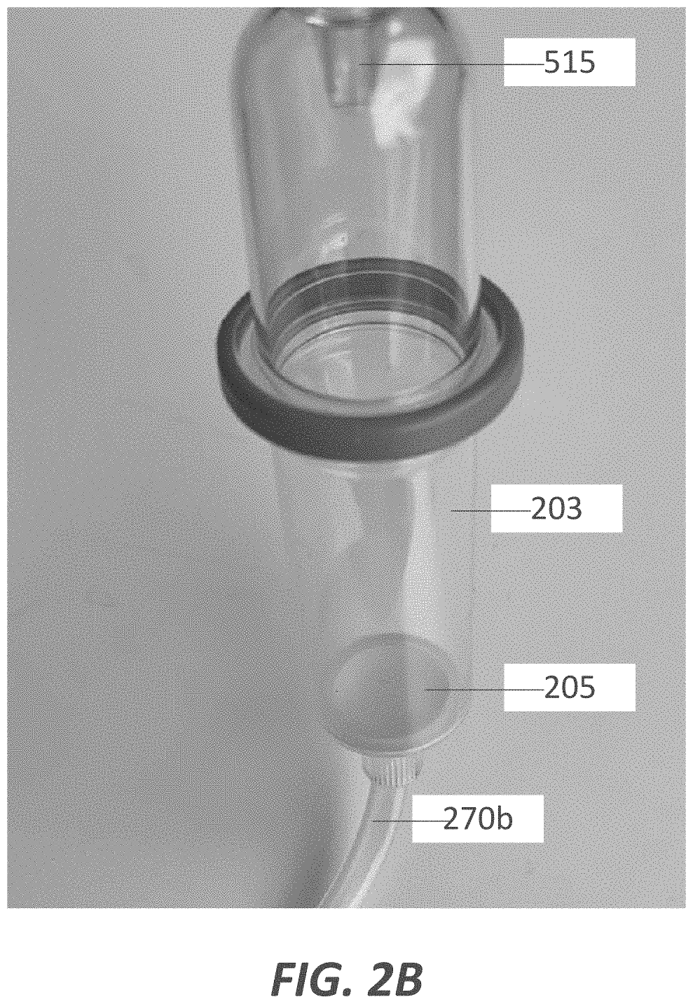

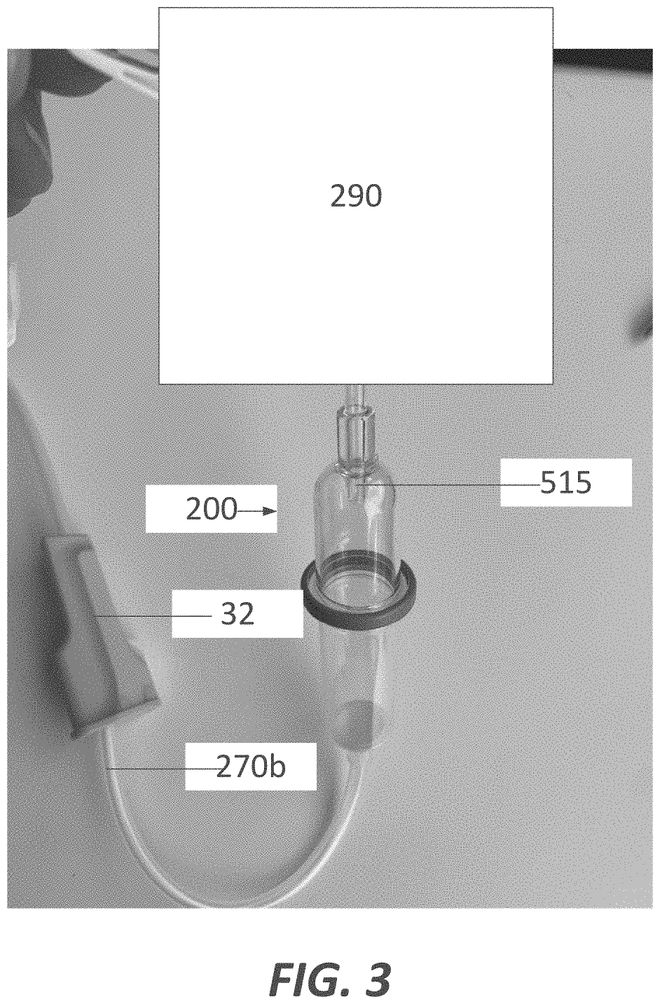

FIGS. 2A-3 show close-up views of the drip chamber 200 in accordance with illustrative embodiments of the invention. Fluid enters the drip chamber 200 from the IV source bag 290 via a fluid inlet 201, which is part of a drip chamber cover 202. Coupled with the drip chamber cover 202 is the pneumatic tube 26. A transparent drip chamber body 203 is coupled (e.g., bonded) to the drip chamber cover 202 at its proximal end and coupled (e.g., bonded) to IV tube 270b at its distal end. A hydrophilic filter membrane 205 is positioned at the bottom of the drip chamber body 203 to permit liquid to pass while preventing air from passing into the tubing 270b. FIG. 3 shows close-up view of the drip chamber 200 and the roller clamp 32, providing a proportional flow resistance through the IV tubing 270 in accordance with illustrative embodiments. From this view, a molded drop former 515 can be seen. The drop former 515 is discussed later.

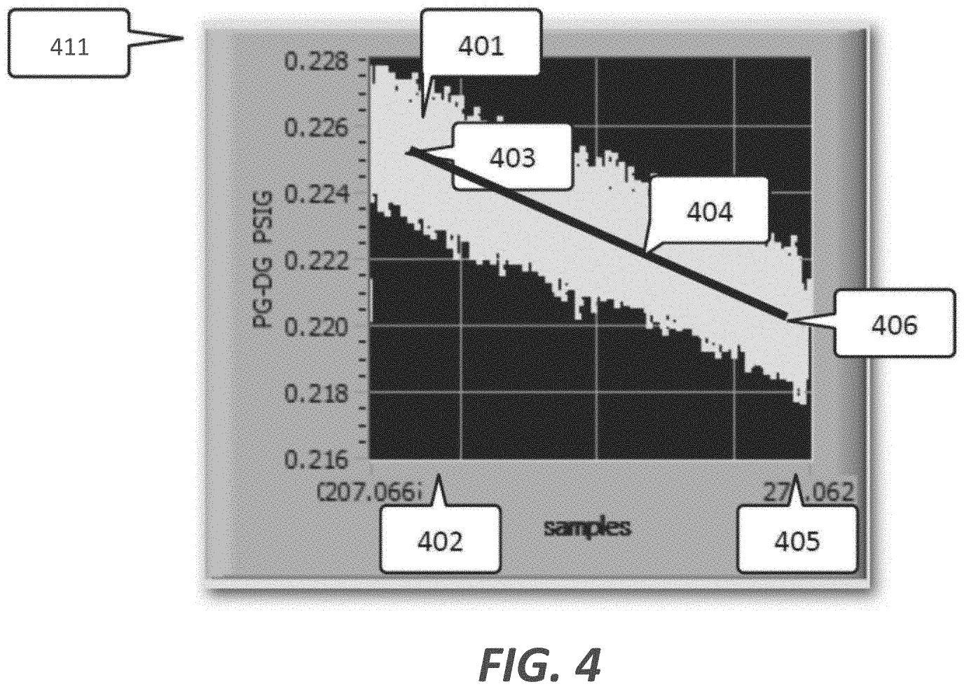

FIG. 4 shows a chart 411 of pressure signals over time, recorded from pressure within the pneumatic tube 26. Pressure signals 401 can be seen as having relatively high frequency oscillations. At initial time 402, the average initial pressure 403 can be recorded, illustrated in this example as approximately 0.225 PSIg. The long term pressure change is shown by a curve fit line 404. At final time 405, the average final pressure 406 can be recorded, illustrated in this example as approximately 0.220 PSIg. Long term pressure decreases (e.g., average final pressure 406) represents the decrease in pressure due to a decreasing head height of source bag 290.

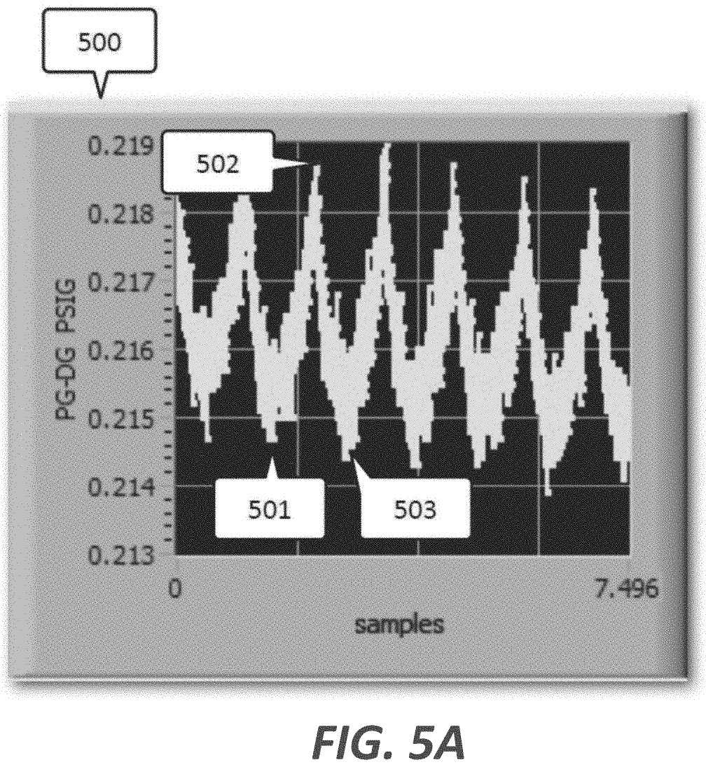

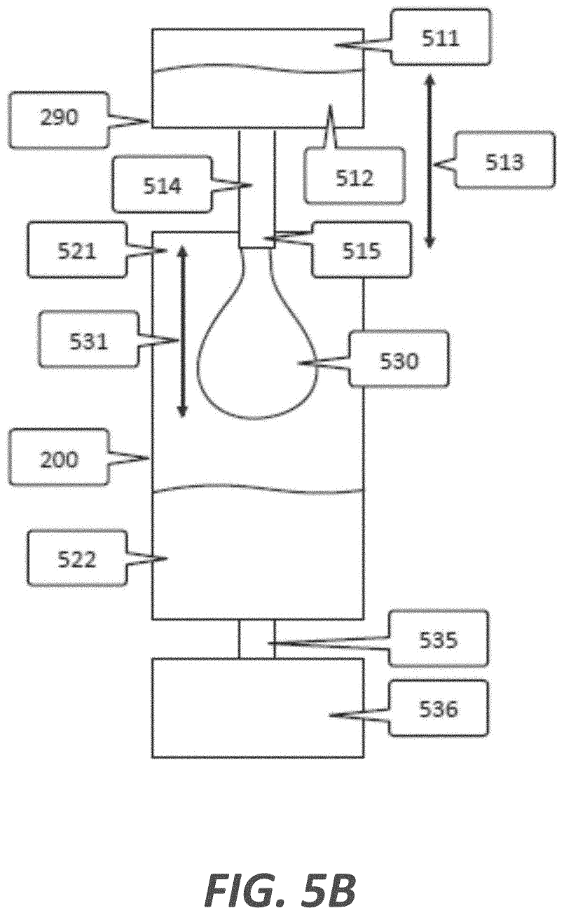

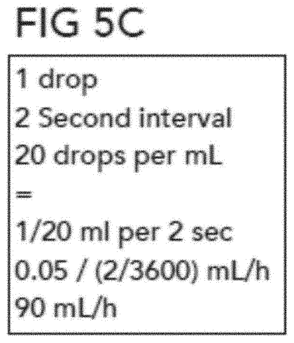

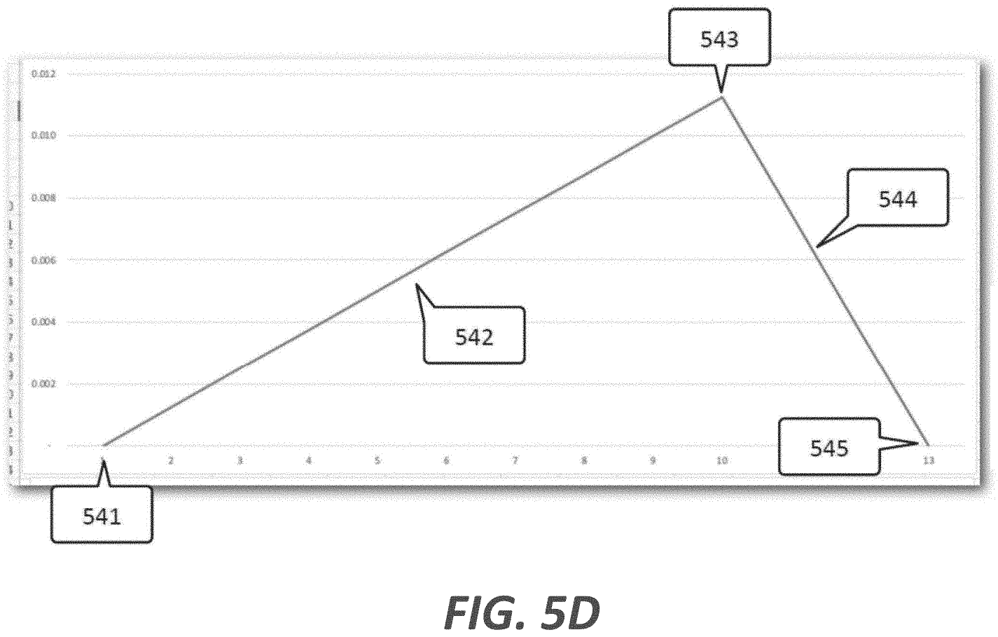

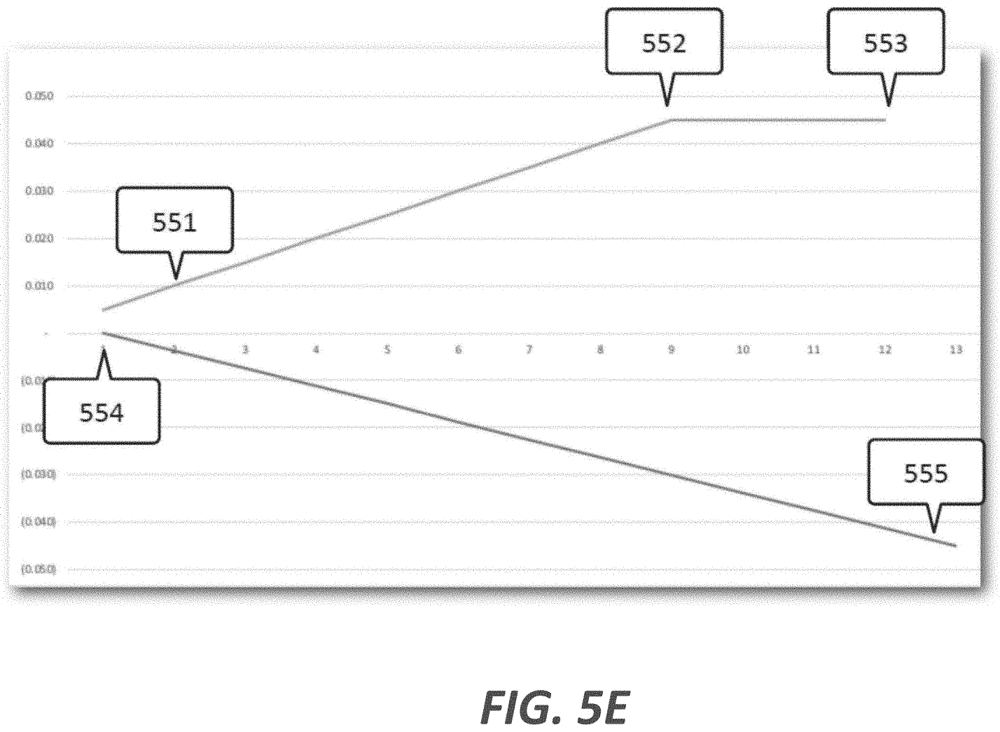

FIG. 5A shows a chart of pressure signals over time, recorded from pressure within pneumatic tube 26. Short term pressure change 500 represents the change in pressure within the drip chamber 200. A Local Minimum Pressure 501 can be recorded, illustrated in this example as approximately 0.2147 PSIg. A local maximum pressure 502 can be recorded, illustrated in this example as approximately 0.2186 PSIg. A second local minimum pressure 503 can be recorded, illustrated in this example as approximately 0.2145 PSIg. The time interval between events 501 and 503 represents the period between drops.

FIG. 5B schematically illustrates the derivation of pressure changes with drop formation. A fluid container 290 has a liquid volume 512 and a gas volume 511. An inlet conduit 514 terminates in a molded drop former 515. The drip chamber 200 has a chamber gas volume 521 and a chamber liquid volume 522. Liquid exits the drip chamber 200, traveling through an outlet conduit 535 and to the patient 536 (e.g., via tubing 270 omitted in this figure). As a drop 530 forms within chamber gas volume 521, the surface tension of the drop 530 allows the formation of a column of liquid equal to the drop height 531, which can be considered to add to the source 290 head height 513. This further increases the drop formation within the chamber gas volume 521. The volume of the drop 530 serves to compress the air in chamber gas volume 521 causing an increase in pressure that is detectable by the pressure sensor 462. When the drop 530 is “pinched off,” the drop height 531 goes to zero and, a meniscus is formed at the molded drop former 515, effectively stopping the flow of the liquid volume 512.

FIG. 5C illustrates a flow rate calculation using drop intervals as discussed above. Illustrative embodiments may determine an interval between drop formation, and use the known volume of the drop to determine a flow rate into the chamber 200. As an example, if a drop 530 is observed with a 2 second interval, then the flow rate can be calculated to be approximately 90 mL/h (assuming a 20 drop per mL configuration). The volume of an individual drop is determined by the molded geometry of the drop former 515.

FIG. 5D illustrates a blown up and idealized view of FIG. 5A between points 501 and 503. A sawtooth pattern is observed. An initial pressure 541 rises as a drop is visibly forming as shown by Rising Pressure 542. At drop falling point 543, the pressure reverses as seen by declining pressure 544 until it reaches final pressure 545. As shown, the initial pressure 541 and the final pressure 545 are approximately equal. This condition represents a state of equilibrium in which the flow of liquid into the drip chamber 200 is equal to the flow out of the drip chamber 200.

FIG. 5E shows an experimentally validated theory of a decomposition of the waveform of FIG. 5D. A quantity of air molecules in the chamber is fixed (e.g., the air 521 in FIG. 5B is trapped between liquid volumes 512 and 522). Thus, in general, the amount of liquid entering the drip chamber 200 (e.g., via inlet conduit 514) is equivalent to the amount of liquid leaving the drip chamber 200 (e.g., via outlet conduit 535). Accordingly, the pressure on the fixed number of gas molecules determines the chamber gas volume 521. Thus, we would in expect that pressure changes within the chamber gas volume 521 would be very slow and slight (e.g., a gradual decay due to a decreasing head height within the bag 290). However, the inventors were surprised to discover that pressure changes within the chamber gas volume 521 experiences a small but detectable pressure spike every time there is a drop (e.g., 3/1000 of a PSI). This is in contrast to the expectation of a steady pressure in the gas volume 521 when the fluid input is equivalent to the fluid output in the drip chamber 200.

Accordingly, the inventors theorized, and have experimentally validated, that the spikes in pressure are a function of two different pressure waveforms shown in FIG. 5E. An initial outflow pressure contribution 554 falls steadily (as fluid leaves the drip chamber 200) during the period shown in FIG. 5E to reach a final outflow pressure contribution 555. This is due to the steady flow of chamber liquid volume 522 through the outlet conduit 535. As liquid leaves, the chamber gas volume 521 expands and pressure drops based on ideal gas law principles. Drop inflow pressure contribution 551 increases as the size of drop 530 in the chamber 200 increases. At drop detachment point 552, the drop inflow pressure contribution 551 stops rising as inflow into the chamber 200 is effectively stopped by the meniscus. Drop inflow pressure contribution 551 thus will not increase until a sufficient differential pressure is created to break the meniscus and start the cycle again as shown in FIG. 5A. The arithmetic sum of the waveforms in FIG. 5E is equal to the observed net pressure pattern in FIG. 5D.

Expressed another way, the inventors discovered that the instant the drop forms and falls off, a substantially flat sheet of liquid having surface tension in the inflow conduit 514 (i.e., the meniscus) stops the flow until there is sufficient pressure differential to start forming the drop. Expressed mathematically, an integral under the curve 554 is equal to an integral under the curves 551 and 553. While the total volume of fluid into and out of the chamber 200 is equivalent, the change in pressure is continuous in 554 and discontinuous in 551 to 553.

The inventors experimentally verified this theory by watching the flow, clamping off the inlet conduit 514 and isolating the impact of the outlet conduit 535 on pressure (e.g., curve 554). Similarly, the inventors clamped off the outlet conduit 535 and isolated the impact of the inlet conduit 514 on pressure (e.g., curves 551 and 553). Thus, the inventors determined that a small but measurable spike in pressure, e.g., when gas volume 521 in the chamber 200 remains the same, could be used to reliably count drops entering the chamber 200. Furthermore, illustrative embodiments may further use this technique to monitor flow, and also a control signal to impose positive pressure in the drip chamber 200 (e.g., from controller 400 sending a signal to pneumatic generator 410). The controller may send an indication of flow rate and/or volume left in the container 290 to the medical practitioner.

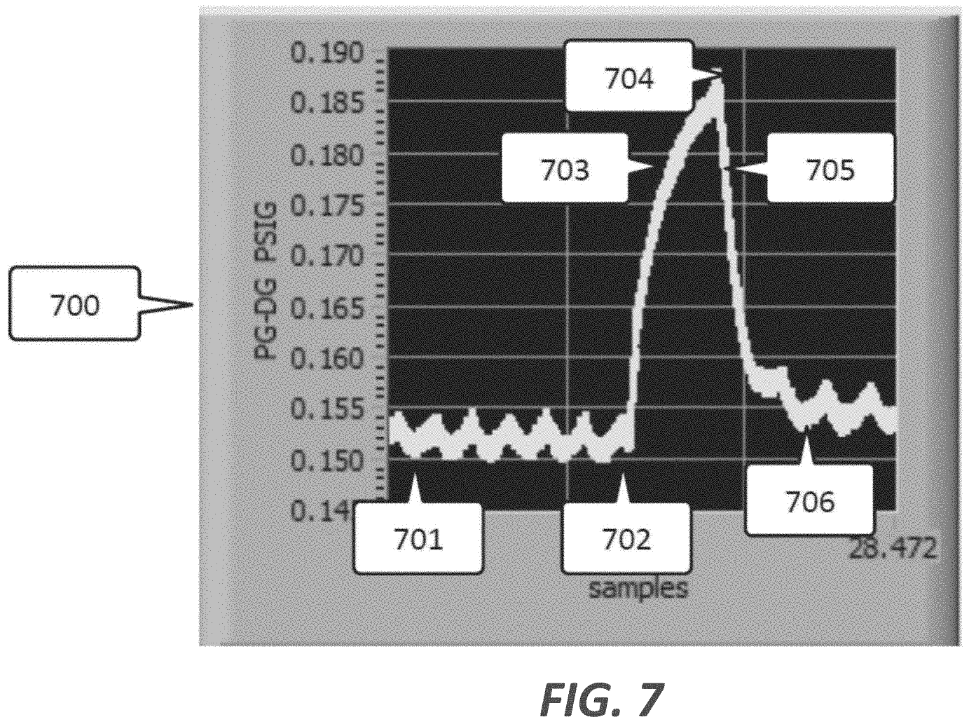

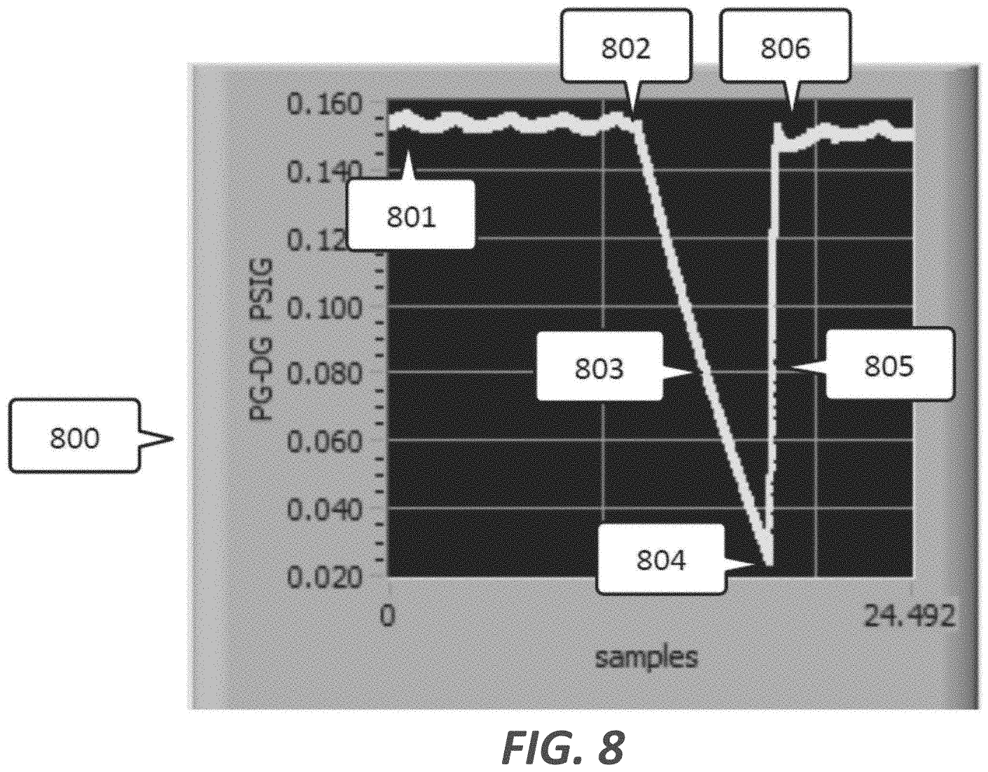

As an additional advantage, illustrative embodiments enable enhanced monitoring that includes determining why drops 530 have ceased (e.g., it is possible to distinguish between upstream occlusions and downstream occlusions from the perspective of the drip chamber 200), as discussed below with reference to FIGS. 7 and 8.

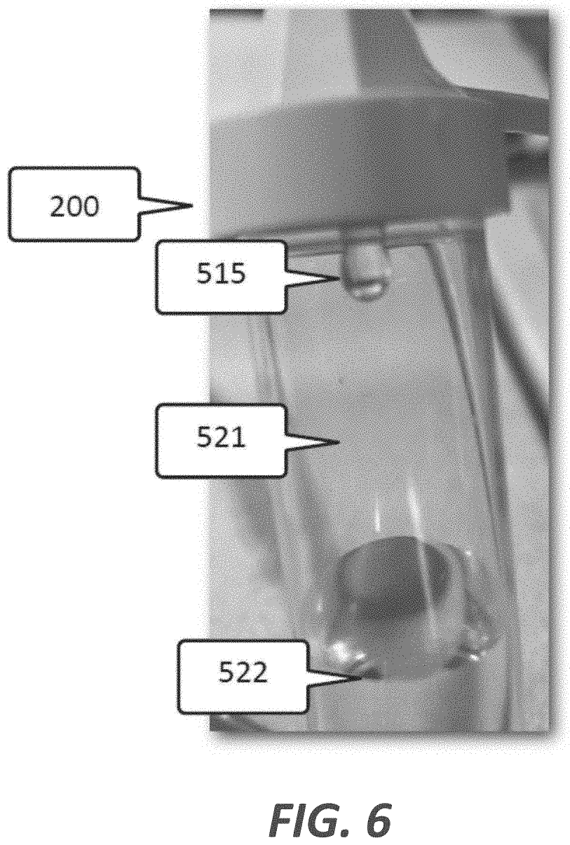

FIG. 6 shows a close-up view of the drip chamber 200. In various embodiments, the drop former 515 is a molded feature, shown with a drop in mid formation. Drop former 515 is constructed in a way to give a uniform drop size, nominally 15 drops per mL in this example. The drops fall into a contained air space (e.g., gas volume 521) and land into a liquid level (e.g., the liquid volume 522) within the drip chamber 200.

FIG. 7 illustrates the pressure response to a downstream occlusion in accordance with illustrative embodiments of the invention. Graph 700 illustrates the pressure recorded from the pneumatic tube 26 over time. Between an initial time 701 and a downstream occlusion time 702, the observed pressure shows oscillation from drop formation and described with reference to FIG. 5A. At the downstream occlusion time 702, the flow path downstream from the drip chamber 200 is fully occluded. Increasing pressure 703 increases until it reaches the equilibrium pressure 704, which is determined by the static head height of source bag 290. In this example, the occlusion is released once Equilibrium pressure 704 has been reached and decreasing pressure 705 reaches the previous pre-occlusion level as a restored oscillating pressure 706. Accordingly, various embodiments may use the pressure signal to determine a downstream occlusion. It should be noted that the occlusion spike may be considered to be on a macro scale (e.g., on the order of 1/10 or 1/100 of a PSI), whereas the drop spike may be considered to be on a micro scale (e.g., on the order of 1/1000 of a PSI). Furthermore, illustrative embodiments may use the pressure signal as a control signal to provide a positive pressure in the chamber 200 and/or to generate an alarm and/or notify a medical practitioner of the occlusion and of the type of occlusion (e.g., upstream, downstream).

FIG. 8 illustrates the pressure response to an upstream occlusion in accordance with illustrative embodiments of the invention. Graph 800 illustrates the pressure recorded from the pneumatic tube 26 over time. Between initial time 801 and upstream occlusion time 802, the observed pressure shows oscillation from drop formation and described in FIG. 5A. At upstream occlusion time 802, the flow path upstream from the drip chamber 200 is fully occluded. Decreasing pressure 803 decreases until it reaches the equilibrium pressure 804, which is determined by the static head height of the patient 536. In this example, the occlusion is released once the equilibrium pressure 804 has been reached and increasing pressure 805 reaches the previous pre-occlusion level as a restored oscillating pressure 806. Accordingly, various embodiments may use the pressure signal to determine an upstream occlusion. It should be noted that the occlusion spike may be considered to be on a macro scale (e.g., on the order of 1/10 or 1/100 of a PSI), whereas the drop spike may be considered to be on a micro scale (e.g., on the order of 1/1000 of a PSI). Furthermore, illustrative embodiments may use the pressure signal as a control signal to provide a positive pressure in the chamber 200 and/or to generate an alarm. Illustrative embodiments may also trigger an alarm notifying a medical practitioner of the occlusion.

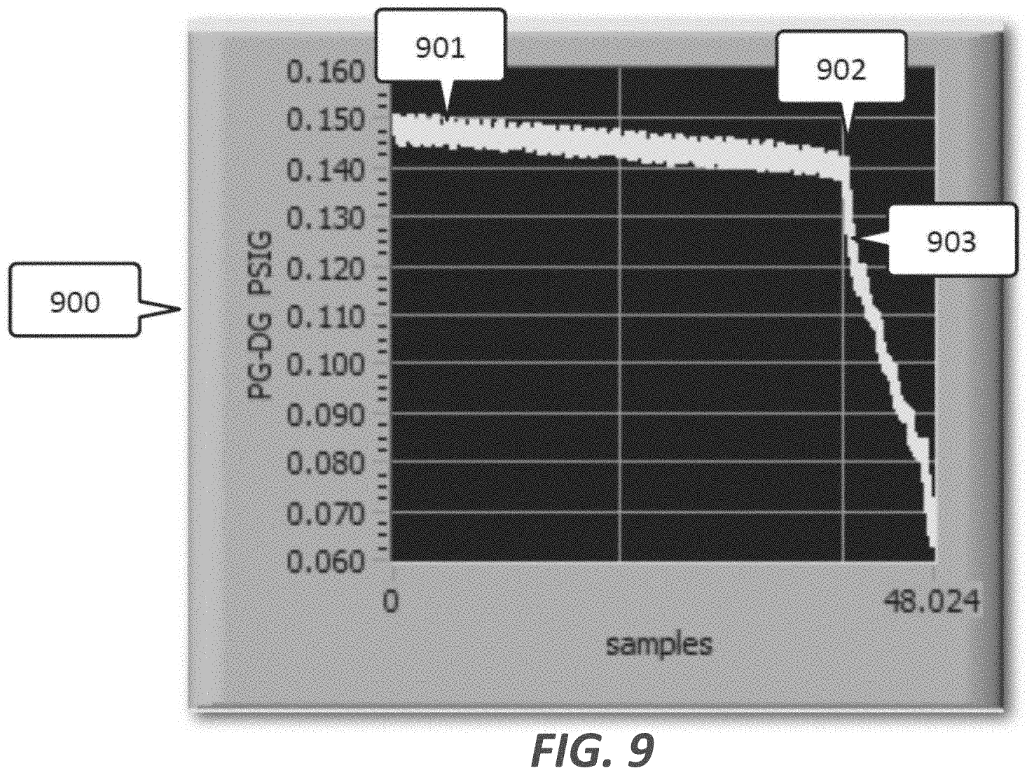

FIG. 9 illustrates the pressure response to the depletion of liquid from the source bag 290. Graph 900 the pressure recorded from pneumatic tube 26 over time. Between initial time 901 and bag empty time 902, the observed pressure shows oscillation from drop formation and described in FIG. 5A. At bag empty time 902, the empty container pressure 903 drops precipitously as a result of rapidly declining head height from a relatively narrow tube. The pressure shown in FIG. 9 is related to the fluid source pressure, but only reaches equilibrium when the flow exiting the drip chamber has been fully stopped. While flow exists, there is a dynamic pressure drop resistance, reducing the recorded pressure in the drip chamber. The combination of the increasing slope due to lower surface area is partially offset by the reduced pressure drop due to slower flow. Nonetheless, the drop in pressure is a definitive pressure pattern for an empty container.

Illustrative embodiments are thus able to determine when the bag 290 is approaching empty. Advantageously, various embodiments may use the pressure measurement as a control signal to adjust infusion rate (e.g., slower so as to not infuse air into the IV line 270). In contrast, systems that rely on optical detectors do not provide any feedback until the bag 290 is empty and drops have stopped forming.

One of skill in the art should understand illustrative embodiments provide a number of advantages including drop counting. Various embodiments advantageously provide insight on upstream fluid from the drip chamber (e.g., fluid input into the drip chamber 200) and downstream fluid from the drip chamber 200 (e.g., fluid output from the drip chamber 200). Illustrative embodiments keep track of the drop count, and thus, enable tracking of the fluid volume in the bag 290 using the pressure in the drip chamber 200.

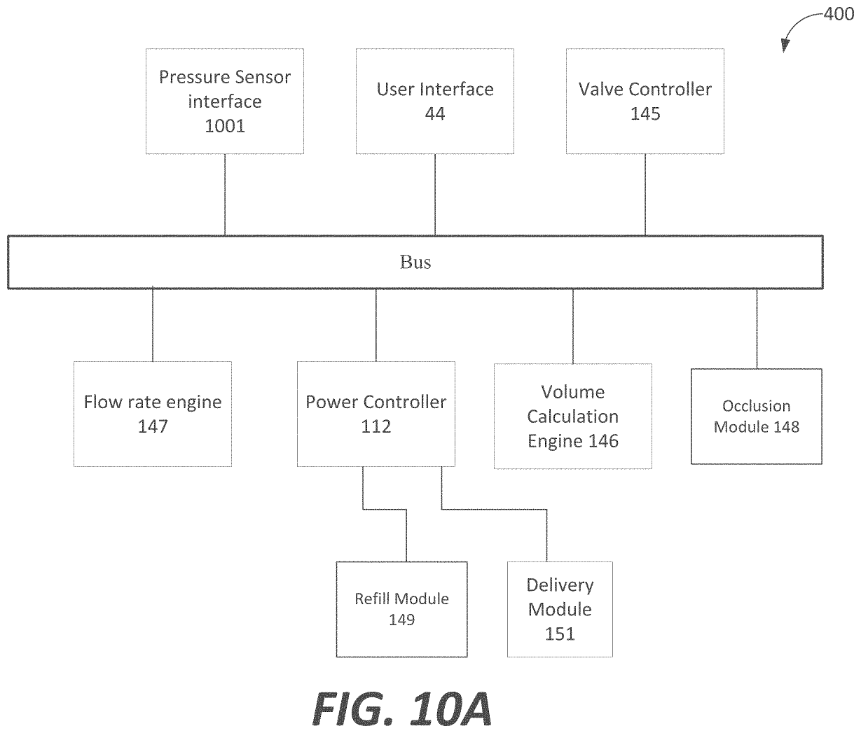

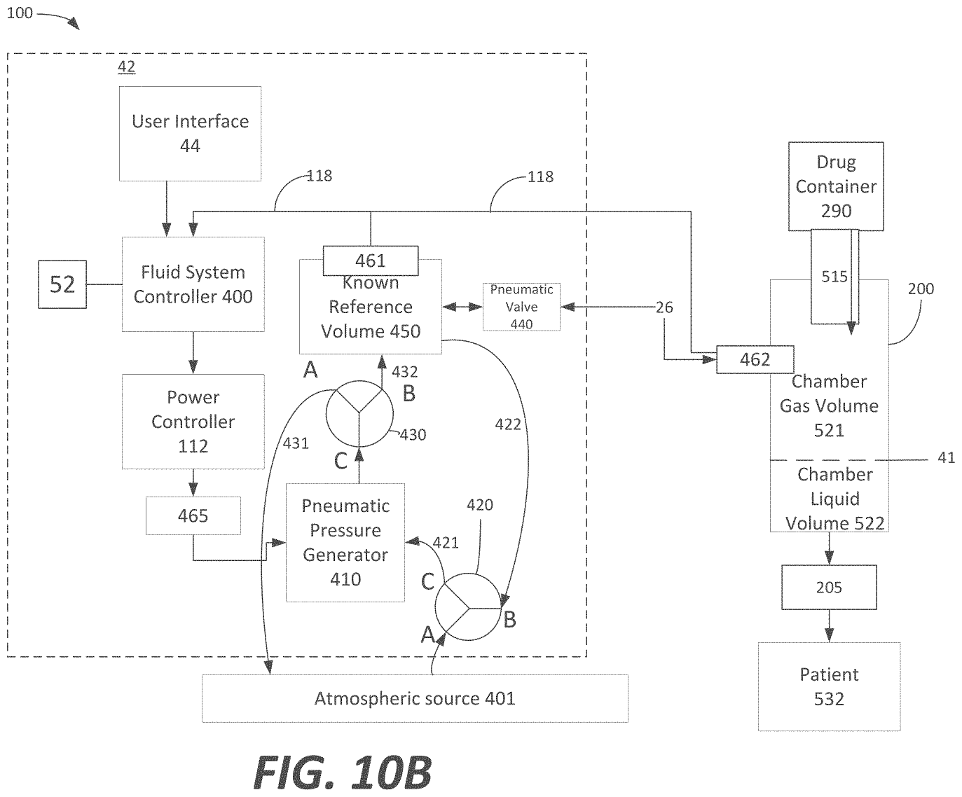

FIG. 10A schematically shows various components of a control system 400 in accordance with illustrative embodiments of the invention. The control system may be within a housing 42. Each of these components is operatively connected by any conventional interconnect mechanism. FIG. 10A simply shows a bus communicating each of the components. Those skilled in the art should understand that this generalized representation can be modified to include other conventional direct or indirect connections. Accordingly, discussion of a bus is not intended to limit various embodiments.

Indeed, it should be noted that FIG. 10A only schematically shows each of these components. Those skilled in the art should understand that each of these components can be implemented in a variety of conventional manners, such as by using hardware, software, or a combination of hardware and software, across one or more other functional components. For example, a volume calculation engine 146 may be implemented using a plurality of microprocessors executing firmware. As another example, the power controller 112 may be implemented using one or more application specific integrated circuits (i.e., “ASICs”) and related software, or a combination of ASICs, discrete electronic components (e.g., integrated circuits), and microprocessors. Accordingly, the representation of the power controller 112 and other components in a single box of FIG. 10A is for simplicity purposes only. In fact, in some embodiments, the power controller of FIG. 10A is distributed across a plurality of different components—not necessarily within the same housing or chassis.

It should be reiterated that the representation of FIG. 10A is a significantly simplified representation of an actual fluid system controller 400. Those skilled in the art should understand that such a device has other physical and/or functional components, such as central processing units, other packet processing modules, and short-term memory. Accordingly, this discussion is not intended to suggest that FIG. 10A represents all of the elements of the fluid system controller 400. Furthermore, in some embodiments, the fluid controller 400 may omit various components shown in FIG. 10A.

Pressure is recorded in the drip chamber 200 using a sensor 462 (e.g., a pressure sensor 462) and analyzed by a computation block 1002. A level of interaction can be obtained by a user interface 44, providing control and reporting functions. A highly accurate and highly resolved pressure generator 410 can be controlled by a power controller 112. The pressure generator 410 may communicate directly with the drip chamber 200 via a pneumatic valve 440 and the pneumatic tube 26. The pneumatic valve assembly 420, 430, and 440 serves to selectively isolate the pressure generator 410 from the drip chamber 200 and to configure the pressure generator 410 to provide either forward or reverse air flow (using valves 420 and 430). Power controller 112 is configured to control the pressure generator 410 and pneumatic valve assembly 420, 430, and 440.

The controller 400 and/or the pneumatic pressure generator 410 are powered by an energy supply 465, which may be a battery or other known voltage and energy source. The user communicates with the controller 400 via the user interface 44, which may be a touchscreen interface 44 with both audio and visual feedback. In various embodiments, the housing 42 may include an inertial sensor 52 (e.g., an accelerometer and/or gyroscope) to provide information about the orientation of the controller 400 and/or the tubing set 24. In preferred embodiments, the drip chamber 227 is physically coupled with the housing 42. For example, the drip chamber 227 may be physically coupled with the housing 42 (e.g., the drip chamber 227 may fit within a chamber receiving portion 50 of the housing 42, see FIG. 2). Accordingly, the housing 42 and the chamber 227 may move and/or rotate together.

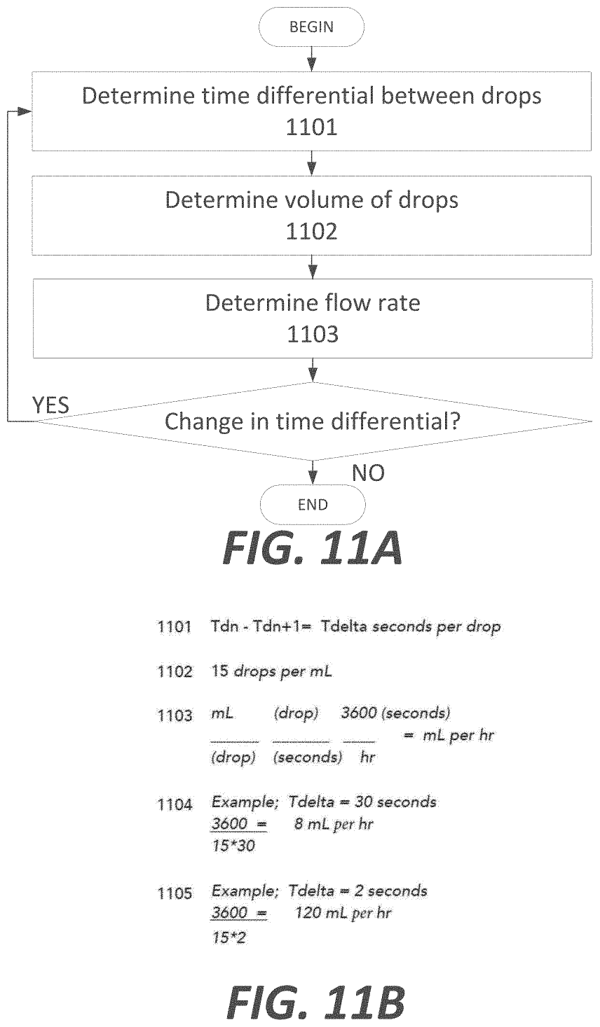

FIG. 11A shows a computational process of the flow rate engine 147 to convert drop detection into a flow rate value in accordance with illustrative embodiments. FIG. 11B shows an example of the process of FIG. 11A. It should be noted that this process may be considered to be simplified from a longer process. Accordingly, the process may have other steps that those skilled in the art likely would use. In addition, some of the steps may be performed in a different order than that shown, or at the same time. Those skilled in the art therefore can modify the process as appropriate. Moreover, as with other figures, many of the structures noted are but one of a wide variety of different structures that may be used. Those skilled in the art can select the appropriate structures depending upon the application and other constraints. Accordingly, discussion of specific structures is not intended to limit various embodiments.

The pneumatic pressure generator 410 provides a precise flow and pressure profile in accordance with the settings of the controller 400. The controller 400 includes at least one microprocessor that can generate a selected power level to actuate the pressure generator 410, causing air to flow through the assembly. The input diverter valve 420 selectively pneumatically couples the pneumatic pressure generator 410 to an atmospheric source 401 or to the reference volume 450. Output diverter valve 430 connects generator 410 to the atmospheric source 401 or to the reference volume 450. The action of the valves 420 and 430 is controllable by the controller 400.

The system 100 enables selective operation of the drip chamber 200. The system 100 may generate a positive pressure in the drip chamber 227. To increase pressure in the chamber 227, pneumatic valve 440 is opened, and gas is joined from the reference volume 450 to the drip chamber 227. To pump fluid from the reference volume 450, the input valve 420 is activated to pneumatically couple common branch C to selection A, allowing flow to come from the atmosphere 401 to the generator 410 via pneumatic connection 421. The output valve 430 is also activated to connect common branch C to selection B, allowing flow to come from the generator 410 to the reference volume 450 via pneumatic connection 432.

To decrease pressure in the drip chamber 200, the pneumatic valve 440 is opened, and fluid (e.g., gas) is pumped out of the drip chamber 227 towards the reference volume 450. To reduce pressure in the reference volume 450, the input valve 420 is activated to connect common branch C to selection B, allowing flow to come from the reference volume 450 to pressure generator 410 via pneumatic connection 422. The output valve 430 is activated to connect common branch C to selection A, allowing flow to come from the generator 410 to the atmospheric source 401 (e.g., ambient air) via pneumatic connection 431.

FIG. 10B schematically shows a detailed block diagram of the system 100 in accordance with illustrative embodiments of the invention. In particular, communications between the controller 400 and the drip chamber 200 are shown. Details of the system are described in U.S. patent application Ser. No. 17/362,603, which is incorporated herein by reference. The system may include a pressure sensor 461 on a reference volume 450. Pressure signals 118 may be sent by a pressure sensor 462 to the fluid system controller 400.

The process begins at step 1101, in which the flow rate engine 147 determines a time differential between drops (i.e., the time differential is the time between a drop and a subsequent drop). The flow rate engine 147 from FIG. 10A uses data from the sensor 462 to compute a time differential Tdelta 1101.

At step 1102, the volume of the drops is determined. Drop size 1200 is a design element of the drip chamber 200 (e.g., the drop former 515), which is provided to the flow rate engine 147. At step 1103, the flow rate is determined. Flow conversion is shown by Example 1104, in which the drop interval is 30 seconds, representing a fluid flow of 8 mL/hr and Example 1105, in which the drop interval is 2 seconds, representing a fluid flow of 120 mL/hr. Thus, the flow rate into the drip chamber 200 is calculated (and out of the drip chamber 200, when fluid volume is held constant in the drip chamber).

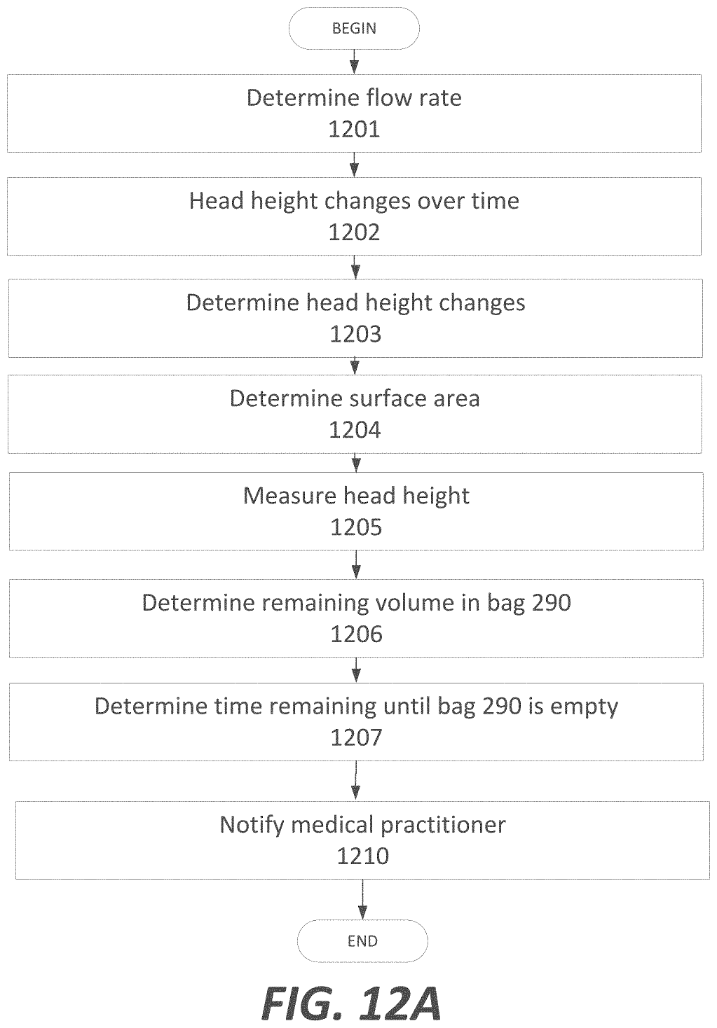

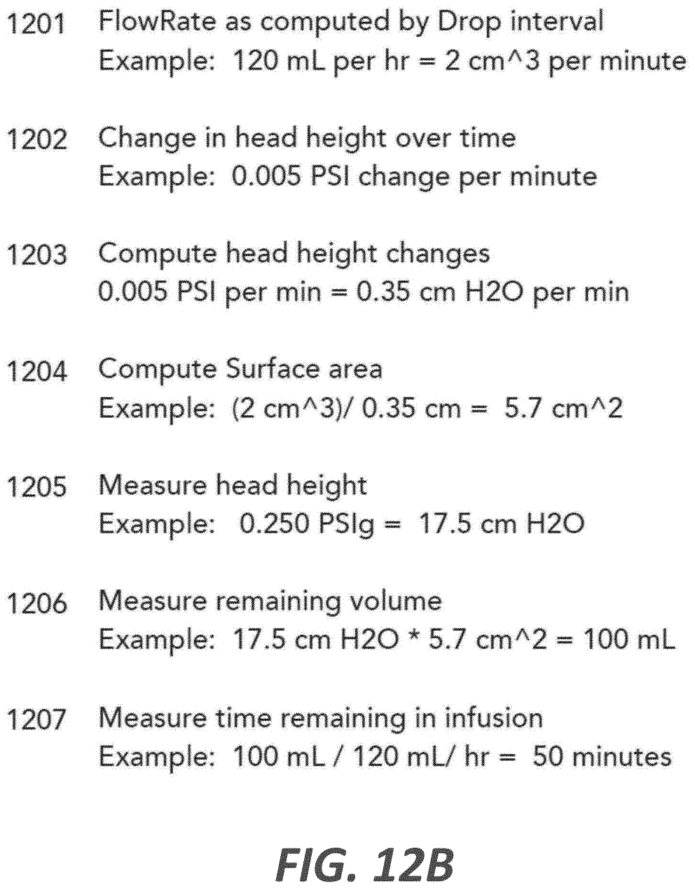

FIG. 12A shows a computational process of the volume calculation engine 146 to determine remaining liquid volume 512 in the bag 290 from a flow rate and a head height in the bag 290 in accordance with illustrative embodiments of the invention. FIG. 12B shows an example of the process of FIG. 12A. It should be noted that this process may be considered to be simplified from a longer process. Accordingly, the process may have other steps that those skilled in the art likely would use. In addition, some of the steps may be performed in a different order than that shown, or at the same time. Those skilled in the art therefore can modify the process as appropriate. Moreover, as with other figures, many of the structures noted are but one of a wide variety of different structures that may be used. Those skilled in the art can select the appropriate structures depending upon the application and other constraints. Accordingly, discussion of specific structures is not intended to limit various embodiments.

At step 1201, a flow rate 1201 is computed as shown in FIGS. 11A-11B. The controller knows the drop size (e.g., 15 drops per mL). If 1,800 drops are counted per hour, that is equal to 120 mL/hr. Therefore, illustrative embodiments count the drops and determine the flow rate.

At step 1202, Head Pressure Change 1202 is measured as shown in FIG. 4. The head pressure is the pressure exerted by the fluid in the bag 290 on the chamber of the drip chamber 200. For the sake of discussion, the controller may determine that pressure is declining 0.005 PSI per minute.

At step 1203, head height changes are derived from the specific gravity of the N fluid, nominally that of H2O, but set to any known value. The volume calculation engine 146 knows the flow rate by counting the drops, as described previously. The volume calculation engine may convert PSI to cm H2O. These are both measurements of pressure, the later representing the weight of the water on earth at sea level.

At step 1204, a surface area of the source 290 liquid is computed by looking at changes in head height vs flow rate. The controller knows the flow rate (e.g., cm\hat{ }3 per minute) and the pressure change over time (e.g., cm H2O per minute). By dividing these, as shown in the example of FIG. 12B, this yields a surface area (e.g., in cm\hat{ }2).

At step 1205, the inlet pressure is measured to determine the head height. This may be taken from a reading as shown in FIG. 4. The remaining volume 1206 in the bag 290 is the product of the head height from step 1205 and the surface area 1204 (measured as cm\hat{ }3 H2O). After the controller determines the volume and the flow rate, at step 1207 the controller may determine the time remaining until the bag 290 is empty. The remaining time is determined by dividing the remaining volume from step 1206 by the flow rate from step 1201. Optionally, a medical practitioner may be notified (e.g., by alarm through the user interface or on an electronic device) of the time remaining until the bag 290 is empty.

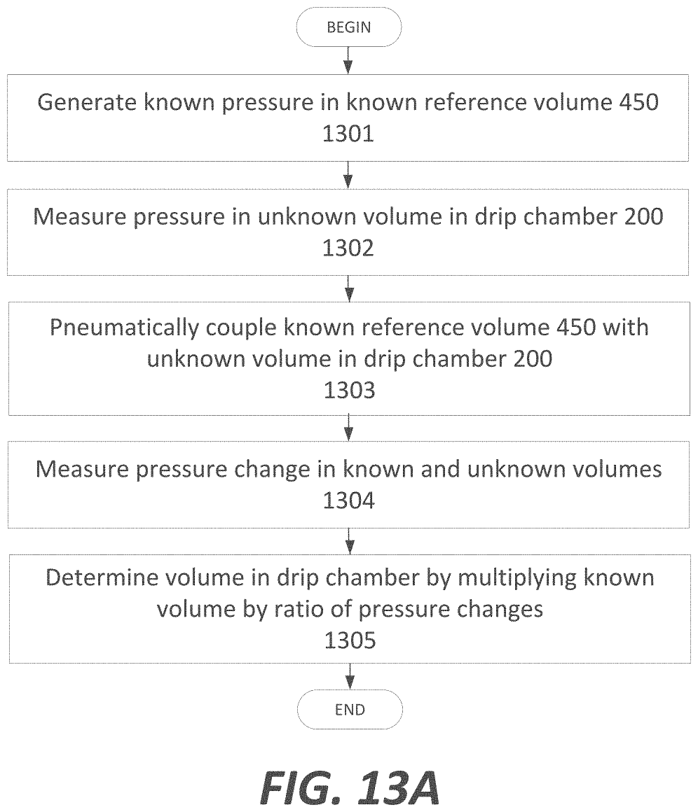

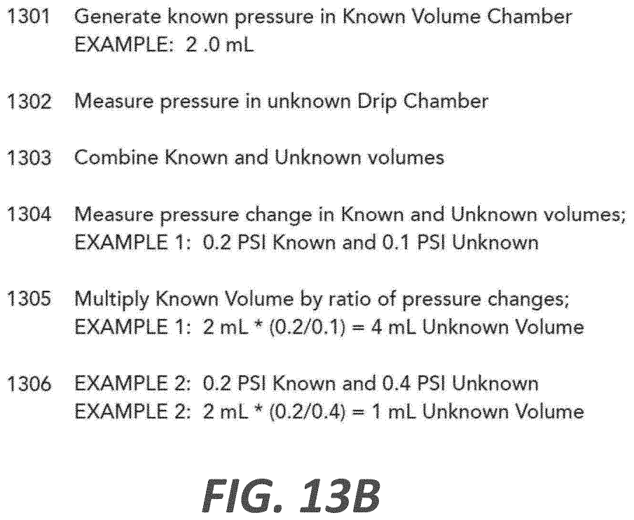

FIG. 13A shows a computational process of the volume calculation engine 146 to determine remaining gas volume 521 in the drip chamber 200 in accordance with illustrative embodiments of the invention. FIG. 13B shows an example of the process of FIG. 13A. It should be noted that this process may be considered to be simplified from a longer process. Accordingly, the process may have other steps that those skilled in the art likely would use. In addition, some of the steps may be performed in a different order than that shown, or at the same time. Those skilled in the art therefore can modify the process as appropriate. Moreover, as with other figures, many of the structures noted are but one of a wide variety of different structures that may be used. Those skilled in the art can select the appropriate structures depending upon the application and other constraints. Accordingly, discussion of specific structures is not intended to limit various embodiments.

The process of FIG. 13A may easily be adapted to determine the volume of the liquid 522 in the drip chamber 200. In various embodiments, the volume calculation engine 146 uses the illustrates the use of Ideal Gas Laws to compute the air volume 521 in drip chamber 200.

The process begins at step 1301, which generates a known pressure in a known reference volume 450 (see FIG. 10B, and application incorporated by reference). Step 1302 determines a known pressure in an unknown volume such as the chamber gas volume 521 (e.g., using pressure sensor 462). Step 1303 pneumatically couples the known and unknown volumes. Step 1304 determines the pressure change in each chamber (e.g., using pressure sensor 462 and/or 461). This measurement can be made equally well with absolute or gauge or differential pressures, since the difference in pressures is used. At step 1305, the volume calculation engine 146 uses the inverse relationship between pressure and volume to compute the unknown volume which, in the example of FIG. 13B is 2× the size of the known volume 450. Example 1306 uses the inverse relationship between pressure and volume to compute the unknown volume 521 which, in this example is ½× the size of the known volume 450.

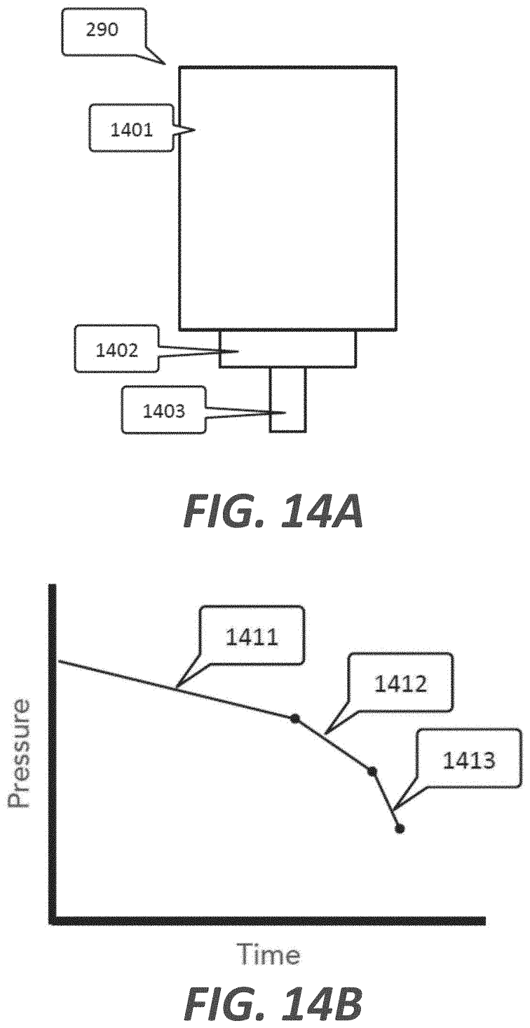

FIG. 14A schematically shows a fluid source container 290 with large diameter 1401 and correspondingly large surface area, such that fluid leaving the container 290 produces a relatively small slope of pressure 1411 shown in FIG. 14B. Medium diameter 1402 and correspondingly medium surface area are configured such that fluid leaving the container 290 produces a medium slope of pressure 1412. Small diameter 1403 and correspondingly small surface area are configured such that fluid leaving the container 290 produces a steep slope of pressure 1413. In practice, a fluid source container 290 could have any arbitrary profile of diameters. FIG. 14B is a representative case in which the negative slope increases quickly as the fluid source container 290 empties.

In illustrative embodiments, a reusable device, referred to as a “pneumatic flow controller,” can be configured to provide a simple function of monitoring drop formation into a drip chamber using pressure signals to detect and count timing intervals. These signals consequently can be used to calculate fluid flow rate in conventional units of measure, such as mL per hour. A variety of disposable, sterile fluid pathway configurations can be fitted with the controller 400. Preferred embodiments provide a configuration of minimal complexity, providing basic functionality.

To that end, the standard the gravity drip administration set 24 is modified with the addition of the pneumatic tube 26 as shown in FIG. 2. The tube 26 also is connected to the pressure sensor 462 and the valve assembly 420, 430, and 440 for the selective application of both positive and negative pressure.

During use, a user may adjust the roller clamp in a conventional fashion, based on the time interval between drop formations in the drip chamber. The pressure within the drip chamber will vary as drops are formed and released, as seen in FIG. 5A. Controller 400 can measure the time interval of the drops and, as shown in FIG. 11A, can compute the flow rate, which can be displayed on the user interface 44.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, programmable analog circuitry, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

Illustrative embodiments may be implemented as a kit, as a complete system, or as a partial system. When implemented as a kit, the items may be packaged in one or more sterile bags and/or packages.

Disclosed embodiments, or portions thereof, may be combined in ways not listed above and/or not explicitly claimed. In addition, embodiments disclosed herein may be suitably practiced, absent any element that is not specifically disclosed herein. Accordingly, the invention should not be viewed as being limited to the disclosed embodiments.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A monitoring system for a gravity infusion IV tubing, the system comprising:
a drip chamber having an inlet configured to receive fluid from a fluid source and an outlet configured to deliver fluid towards a patient;
a pressure sensor pneumatically coupled with the drip chamber, the pressure sensor configured to measure the pressure inside the drip chamber;
a controller configured to receive pressure measurements from the pressure sensor and to use the pressure measurements to count a number of drops entering the drip chamber from the fluid source.

2. The monitoring system of claim 1, further comprising:
an air blocking membrane distal to the drip chamber, the air blocking membrane configured to mitigate air from passing through the air blocking membrane and entering the patient line.

3. The monitoring system of claim 1, wherein the drip chamber has a cover with a pneumatic tube coupling the drip chamber with the pressure sensor.

4. The monitoring system of claim 1, wherein each drop corresponds to a repeating cycle of pressure measurements.

5. The monitoring system of claim 1, wherein the controller is configured to determine an occlusion in the fluid source proximal to the drip chamber from the pressure measurements.

6. The monitoring system of claim 1, wherein the controller is configured to determine an occlusion in the IV tubing distal to the drip chamber from the pressure measurements.

7. The monitoring system of claim 1, wherein the pressure sensor has a sensitivity of at least 1/1000 PSI.

8. The monitoring system of claim 1, wherein the controller is configured to determine a flow rate into drip chamber using the pressure measurements.

9. The monitoring system of claim 1, wherein the controller is configured to determine a flow rate out of the drip chamber using the pressure measurements.

10. The monitoring system of claim 1, wherein the controller is configured to determine a change in relative position of a head height of the fluid in the container relative to the patient.

11. The monitoring system of claim 1, wherein the controller is configured to determine a remaining time of the infusion as a function of the remaining liquid to be delivered.

12. A method of monitoring a gravity infusion IV tubing, the method comprising:
providing:
a drip chamber having an inlet configured to receive fluid from a fluid source and an outlet configured to deliver fluid towards a patient;
a pressure sensor pneumatically coupled with the drip chamber, the pressure sensor configured to measure the pressure inside the drip chamber;
a controller configured to receive pressure measurements from the pressure sensor;
determining a number of drops that enter the drip chamber from the fluid source by detecting a small pressure increase followed by a small pressure decrease.

13. The method as defined by claim 12, further comprising determining a flow rate out of the drip chamber as a function of the pressure measurements.

14. The method as defined by claim 13, further comprising providing an alarm when the determined flow rate deviates by more than a pre-determined amount from a selected flow rate.

15. The method as defined by claim 12, further comprising:
determining a head height of the drug container.

16. The method as defined by claim 15, further comprising:
providing a warning if an empty drug container condition is imminent;
and/or providing an indication of a time left until the drug container is empty.

17. The method as defined by claim 12, further comprising:
providing a positive pressure to the drip chamber to prevent further fluid from flowing from the drug container into the drip chamber, wherein the positive pressure is low enough not to pass through an air-blocking membrane distal to the drip chamber.

18. A computer program product for use on a computer system for monitoring liquid delivery, the computer program product comprising a tangible, non-transient computer usable medium having computer readable program code thereon, the computer readable program code comprising:
program code for causing a pressure sensor pneumatically coupled with a drip chamber to measure pressure within the drip chamber;
program code for determining that a drop is formed in the chamber as a function of the measured pressure;
program code for calculating a flow rate into the chamber as a function of the measured pressure; and
program code for calculating a flow rate out of the chamber as a function of the measured pressure.

19. The computer program product of claim 18, further comprising:
program code for determining an upstream and/or downstream occlusion.

20. The computer program product of claim 18, further comprising:
program code for causing a pressure generator to generate a positive pressure in the drip chamber in response to determining that the drug container is emptying within a pre-defined time period, the positive pressure being sufficient to block fluid flow from the drug container and low enough to not pass through an air-blocking membrane.
